(19) United States
(12) Patent Application Publication
(10) Pub. No.: US 2020/0238047 A1
(43) Pub. Date: Jul. 30, 2020

(54) CATHETER FOR SENSING PRESSURE APPLIED TO FRONT END THEREOF BY USING OPTICAL FIBER AND CATHETER SYSTEM THEREFOR

(71) Applicants: THE ASAN FOUNDATION, Seoul (KR); UNIVERSITY OF ULSAN FOUNDATION FOR INDUSTRY COOPERATION, Nam-gu, Ulsan (KR)

(72) Inventors: Chang Mo HWANG, Seoul (KR); Young Hak KIM, Seoul (KR); Gi Byoung NAM, Seoul (KR); Jae Soon CHOI, Seoul (KR); Gi Seok JEONG, Seoul (KR); So Yeon JIN, Cheongju-si Chungcheongbuk-do (KR)

(21) Appl. No.: 16/635,953

(22) PCT Filed: Aug. 1, 2018
(86) PCT No.: PCT/KR2018/008750

§ 371 Date: Jan. 31, 2020

(30) Foreign Application Priority Data
Aug. 2, 2017 (KR) 10-2017-0098006

(51) Int. Cl. A61M 25/00; A61B 90/00

(52) U.S. Cl. CPC A61M 25/00; A61B 90/06; A61B 18/1492; A61B 2562/0266; A61M 2025/0001; A61B 2090/065

(57) ABSTRACT

According to the present invention, it is possible to provide a catheter capable of measuring the magnitude and direction of an external force applied to the front end of the catheter with a precise sensitivity by using information regarding the amount of change in the amount of light received by the optical fiber inside the catheter body.

### TECHNICAL FIELD

Example embodiments relate to a catheter for sensing a pressure applied to a front end thereof using an optical fiber, and a catheter system including the same. More specifically, example embodiments relate to a catheter of capable of sensitively measuring a magnitude and a direction of a contact force applied to a front end thereof using change of a light-amount and thus suitable for cardiovascular intervention, and to a catheter system including the same.

### BACKGROUND ART

In general, a catheter is a medical device used to insert a tube into a patient's body to treat an affected area using a high frequency, or to inject a medical substance into the body and to discharge a body fluid from the body to an outside thereof.

In performing a surgical procedure using the catheter, when a tip as a front end of the catheter exerts an excessive pressure on the affected portion of the patient, the affected portion may be damaged. On the contrary, when the front end of the catheter contacts the affected area while too little pressure is applied thereto, the affected portion may not be treated properly. Therefore, the pressure applied to the affected portion needs to be precisely measured based on a location and a type of the surgical procedure.

In one example, a treatment and surgical procedure via inducing the catheter a target affected portion using an imaging device is referred to as an interventional surgical procedure. The interventional surgical procedure is characterized by a minimal invasion, which increases safety of the surgical procedure, results in excellent patient prognosis, minimizes a pain and a scar, and thus leads to a high patient satisfaction and has an increased scope of applications thereof. However, the interventional surgical procedure requires precise manipulation of a medical practitioner during the surgical procedure. Thus, success or failure of the procedure depends on experience and ability of the medical practitioner. Further, in treatment of a sensitive area such as a cardiovascular vessel depending on a type of the surgical procedure, the medical practitioner may fail to precisely position the catheter, resulting in damage to the vessel. Further, the interventional surgical procedure may cause other complications, radiation exposure, and the like. Thus, a medical device and equipment to enable precise and accurate surgical procedures in a short time is urgently needed. In other words, in order to minimize the complications of the patient depending on the experience and ability of the medical practitioner and in order to avoid the continuous radiation exposure of the medical practitioner as a number of patients are treated, configuring a control system to remotely perform the interventional surgical procedure is recognized as a major technical issue.

In order to configure the remote control system for a cardiovascular intervention procedure, main hardware components of the system may include a catheter to be guided to a heart for the interventional surgical procedure, a master (haptic master manipulator) to allow the medical practitioner to operate the catheter, and a slave (slave robot) that controls the catheter in conjunction with the manipulation of the master. In this connection, the catheter deliveries a stent or has an electrode for high frequency ablation to perform radiofrequency catheter ablation. As mentioned above, the catheter requires the precise control thereof. When the medical practitioner controls remotely the catheter, functional precisions of sensing information, location information, electrocardiogram information released to the catheter will directly affect the success or failure of the surgical procedure.

In the cardiovascular intervention, the catheter enters the heart and contacts the heart's inner wall to map the heart. In the cardiovascular intervention, it is particularly important that a magnitude and direction of a contact pressure onto a front end of the catheter be precisely measured. When RF is applied while the catheter is not in contact with a target tissue during the radiofrequency catheter ablation, a blood around the catheter electrode located inside the atrium is solidified to cause a blood clot, leading to cerebral infarction and major organ embolism. To the contrary, the catheter excessively contact the inner wall of the heart where the atrial inner wall is constantly contracting and relaxing, this may cause a major medical accident such as a puncture of the inner wall.

As the precise measurement of the pressure applied to the front end of the catheter during mapping or tissue radiofrequency ablation, various types of sensors have been proposed to measure the pressure applied to the tip of the catheter. Conventionally, a force sensor using an electric based pressure sensing element in which an output current varies based on a force applied externally is used. However, in the force sensor using the electric based pressure sensing element, change of the output current may be small when a small external force is applied. Thus, an expensive equipment is further needed to accurately measure change in the current. Further, when the amount of the current is increased by increasing a size of the electric based pressure sensing element, a size of the catheter increases.

Accordingly, a prior art proposing another solution to measure the pressure applied to the tip of the catheter includes U.S. Pat. No. 8,567,265 disclosing a catheter that senses a force applied to the front end thereof in three axial directions using an optical fiber. FIG. 1 shows the catheter in U.S. Pat. No. 8,567,265. Referring to FIG. 1, the catheter in U.S. Pat. No. 8,567,265 uses not the conventional electric based pressure sensing element but the optical fiber to calculate a flexure and a contact pressure via analysis of Fabry-Perot interferometer resulting from reflection of light occurring when the front end of the catheter is flexed. The catheter in U.S. Pat. No. 8,567,265 as shown in FIG. 1 has a sensing assembly 92 having a structural member 102 having three level gaps 921. In this connection, the structural member 102 has threes slit-shaped gaps 921 having different levels, each gap being defined, by 120°, in an outer circumferential surface thereof. In this connection, three optical fibers 104 are arranged in three positions spaced from each other by 120 degrees so that an output end of an optical core of each optical fiber is located in each gap 921. The three-level gaps 921 forms a spring-like segmented structure. When an external force F is applied to the front end of the catheter in a certain direction, a spacing of each gap 921 at each position may vary. Accordingly, a multi-interference phenomenon of the light thus reflected and received by the optical fiber 104 is analyzed to detect the magnitude and direction of the contact force.

FIG. 2 illustrates a principle of a catheter to which the catheter configuration disclosed in FIG. 1 of U.S. Pat. No. 8,567,265 and is an excerpt from a TactiCath™ product description from St. Jude Medical company. The Fabry-Perot interferometer is generally composed by inserting one resonant layer (gap cavity) between two high reflectivity mirrors. A fundamental principle of the Fabry-Perot interferometer is that when light of multi-wavelengths λ1, λ2, and λ3 transmitted through an optical fiber is incident on a filter, multiple interferences are caused in the resonant layer, thereby transmitting light of only a certain wavelength and reflecting light of other wavelengths, thereby to select desired data. Referring to FIG. 2, the gap 921 of the structural member 102 is shown as a Fabry-Perot cavity. It may be understood that the direction and magnitude of the external force are calculated using wavelength information of the light subjected to multi interferences through the three gaps 921 under the Fabry-Perot interferometer.

Other products to which a configuration of the catheter for sensing the pressure applied to the front end as shown in FIGS. 1 and 2 include ThermoCool SmartTouch catheter from Johnson & Johnson Medical's Biosense Webster. The Thermocool SmartTouch catheter accurately transmits a strength and direction of a contact force of the catheter and improves safety and is approved by USFDA and launched in Korea.

As such, an approach for measuring the pressure applied to the tip of the catheter is being developed from a catheter employing the electrical based pressure sensing element to a catheter employing the optical fiber having excellent safety.

However, the conventional catheter as described with reference to FIG. 1 and FIG. 2 requires the structural member 102 in addition to the optical fiber as the sensing assembly 92. In this connection, in the structural member 102, the slit shaped gaps 921 should be arranged at 120° spacing and be formed to have different levels. That is, the structural member 102 should have at least three gaps 921 equally spaced from each other and arranged in a longitudinal direction of the structural member. Therefore, in the conventional catheter, a length of the structural member 102 may occupy most of a length of the front end of the catheter. Thus, there is a limit in measuring precise displacement of the front end of the catheter. Further, analyzing the wavelength information of light using the multiple interference phenomenon such as the Fabry-Perot interferometer causes a problem that a system design is complicated and a manufacturing cost increases accordingly.

Accordingly, the present applicant has achieved a novel type catheter in which, as in the prior art, the pressure applied to the front end of the catheter is measured using the optical fiber, but the magnitude and direction of the pressure applied to the front end are detected only based on light-amount information that is easily acquired and analyzed.

The related prior art includes U.S. Pat. No. 8,567,265.

### DETAILED DESCRIPTION OF THE INVENTION

### Technical Subject

The present disclosure aims to provide a catheter that measures a pressure exerted on a front end of the catheter based on a change in a light-amount. Further, the present disclosure aims to provide a catheter that may measure a magnitude of a pressure applied to the front end of the catheter based on three-axis directions of the pressure. Further, the present disclosure aims to provide a catheter that may measure a contact force of the front end thereof more precisely because a structure of a sensing assembly for the pressure measurement is simple and thus is formed in a micro region of the front end of the catheter.

### Solutions

According to an aspect, there is provided an catheter including: a catheter body having first and second regions, wherein one or more channels are formed in the first region, while the second region includes a front end having a tip subjected to an external force, wherein a gap is defined between the first region and the second region; an optical fiber including an optical core received in the channel and located in the first region, wherein the optical core emits light through the gap into the second region and then receives light reflected from a reflective mirror; and the reflective mirror located inside the front end and located in the second region, wherein the reflective mirror is positioned such that a spherical face of the mirror is not flat toward the first region, wherein as the external force is applied to the tip, a spacing in the gap between an output end of the optical core and the reflective mirror varies, wherein the catheter senses a direction and a magnitude of the external force applied to the tip based on change in an amount of light reflected from the reflective mirror due to the variation.

Preferably, the catheter in accordance with the present disclosure may further include an elastic member provided to surround the gap and disposed inside the catheter body and made of a material having an elastic force different from an elastic force of the catheter body, such that the elastic member concentrates the external force applied to the tip on the front end.

Preferably, the optical fiber may include three optical cores arranged at an angular spacing of 120°, wherein the three optical cores respectively receive different amounts of light reflected from the reflective mirror displaced laterally as a lateral external force is applied to the tip.

Preferably, the optical fiber may further include an optical filter coated on the output end thereof, wherein the optical filter reflects a portion of light emitted from the optical core into the gap and transmits a remaining portion of the light emitted from the optical core therethrough to the second region.

Preferably, the optical fiber may receive the light reflected from the optical filter as first light, and may receive the light reflected from the reflective mirror through the optical filter as second light, wherein the catheter senses the direction and the magnitude of the external force applied to the tip based on amount information of the second light.
Preferably, the reflective mirror has a spherical face convex toward the first region, wherein when the external force is applied outwardly from an linear axis of the catheter body, light output from the optical core is incident on the reflective mirror at an oblique angle.

According to another aspect, there is provided a catheter system including: a catheter including: a catheter body having first and second regions, wherein one or more channels are formed in the first region, while the second region includes a front end having a tip subjected to an external force, wherein a gap is defined between the first region and the second region; an optical fiber including an optical core received in the channel and located in the first region, wherein the optical core emits light through the gap into the second region and then receives light reflected from a reflective mirror; and the reflective mirror located inside the front end and located in the second region; and a light-amount analyzer to receive an amount of the reflected light received by the optical fiber and to calculate a direction and a magnitude of the external force applied to the tip based on change in the received amount of the light.

Preferably, the optical fiber may further include an optical filter coated on the output end thereof, wherein the optical filter reflects a portion of light emitted from the optical core into the gap and transmits a remaining portion of the light emitted from the optical core therethrough to the second region, wherein the light-amount analyzer receives an amount of first light as the light reflected from the optical filter, and an amount of second light as the light reflected from the reflective mirror through the optical filter and calculates the direction and magnitude of the external force applied to the tip of the catheter based on the amount of the second light.

### Effects

According to example embodiments, a catheter may be provided that may accurately and precisely measure the magnitude and direction of the external force applied to the front end of the catheter using the change information of the amount of the light received by the optical fiber in the catheter body.

More specifically, the catheter according to the present disclosure is configured to measure the external force applied to the front end using only a single gap structure spaced between the first region and the second region at the front end of the catheter body. Therefore, there is an advantage that the sensing assembly may be implemented in a micro region of the front end of the catheter.

Further, the light-amount analyzer in accordance with the present disclosure measures the pressure via analysis of change of the light-amount. The light-amount information is easily acquired and analyzed, thereby to easily design a system for pressure sensing and to reduce a manufacturing cost.

According to example embodiments, the light-amount analyzer may detect the direction in which the pressure is applied based on the light-amount information of the at least three optical cores arranged by a 120°. When the external force is applied to the catheter tip, the reflective mirror is displaced in the application direction of the external force. Thus, the light-amounts received by the three optical cores may be identified in a distinguished manner. In particular, a structure of the reflective mirror has a spherical surface having a curvature, and thus the mirror reflects the output light from the optical core at an oblique angle when the mirror is displaced in a lateral direction. Accordingly, the three optical cores receive remarkably reduced light-amounts when a lateral pressure is applied than when a pressure in a linear axial direction of the catheter is applied, thereby discriminating the direction of the external force.

### BRIEF DESCRIPTION OF DRAWINGS

FIG. 1 illustrates a conventional catheter for sensing a pressure using an optical fiber.

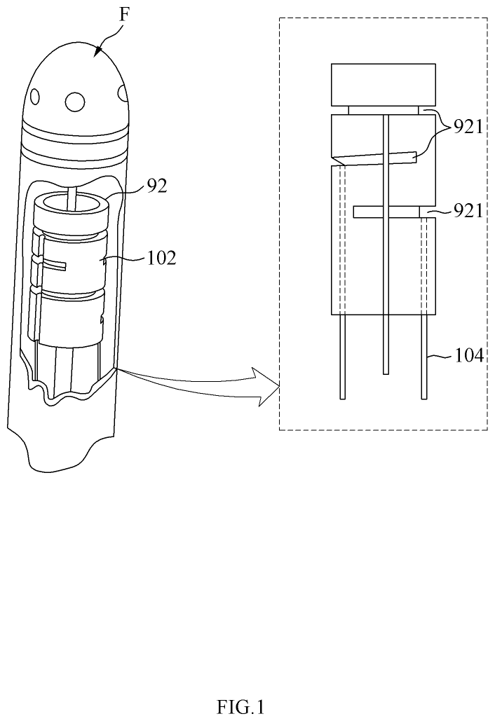

FIG. 2 illustrates a sensing principle of a catheter product for sensing a pressure to which the conventional catheter for sensing a pressure of FIG. 1 is applied.

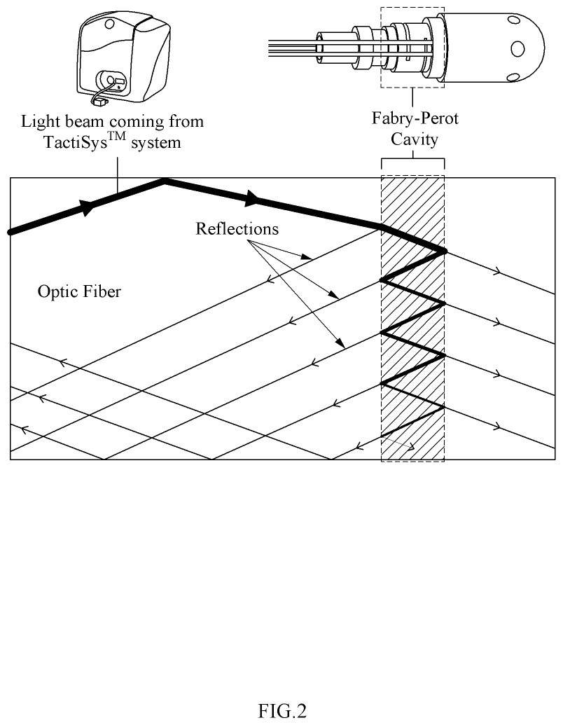

FIG. 3 illustrates a catheter system according to an embodiment of the present disclosure.

FIG. 4 shows an exploded view of an front end of a catheter according to an embodiment of the present disclosure.

FIG. 5 shows an internal structure of an optical fiber of a catheter according to an embodiment of the present disclosure.

FIG. 6 shows an internal structure of an optical fiber when an external force is applied upwardly to a front end of a catheter according to an embodiment of the present disclosure.

FIG. 7 shows an internal structure of an optical fiber according to another embodiment of the present disclosure.

FIG. 8 shows an internal structure of an optical fiber when an external force is applied upwardly to a front end of a catheter according to the embodiment of FIG. 7.

### BEST MODE FOR CARRYING OUT THE INVENTION

Hereinafter, the present disclosure will be described in detail with reference to the accompanying drawings. However, the present disclosure is not restricted or limited to the exemplary embodiments. Like reference numerals in the drawings denote members that perform substantially the same function.

The purpose and effect of the present disclosure may be naturally understood or clarified from the following description. The following description does not limit the purpose and effect of the present disclosure. Further, in describing the present disclosure, when it is determined that detailed descriptions of a well-known component related to the present disclosure may unnecessarily obscure gist of the present disclosure, the detailed description thereof will be omitted.

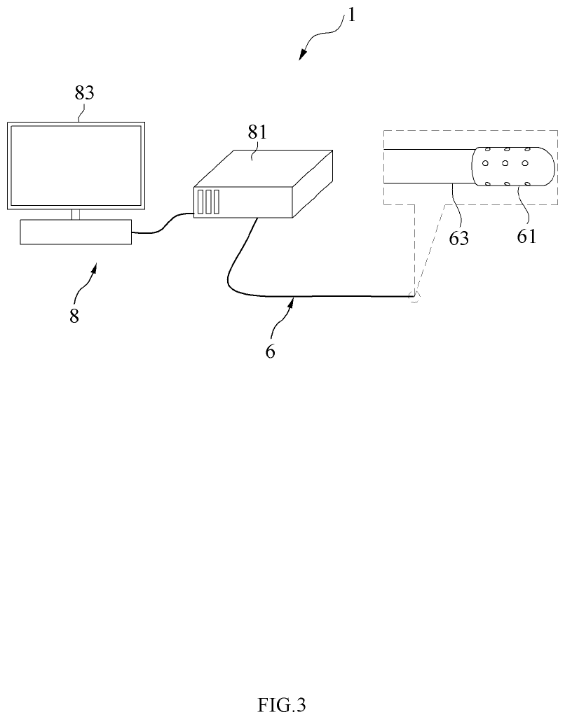

FIG. 3 shows a catheter system 1 according to an embodiment of the present disclosure.

The catheter system 1 according to the present embodiment may include a catheter 6 and a light-amount analyzer 8. The catheter system 1 according to the present embodiment is configured to measure a magnitude and direction of an external force applied to a tip 61 of the catheter 6 to obtain three-dimensional pressure information of the tip 61 in contact with an inner wall of a heart. In the catheter system 1 according to the present embodiment, an optical fiber 65 is implemented as a sensing assembly for measuring the pressure of the tip 61. In this case, the pressure measurement is calculated using amount information of the light received by the optical fiber 65. The catheter system 1 according to the present embodiment may include the catheter 6, and the light-amount analyzer 8 including a processor 81 for quantitatively calculating the change in the light-amount to calculate the magnitude and direction of the external force, and a display 83 for visually implementing the calculated pressure. Hereinafter, a detailed configuration of the catheter 6 according to the present embodiment will be described in detail.

The catheter 6 may include a catheter body 63, the optical fiber 65, the tip 61, and an elastic member 67.

The tip 61 may be implemented in a form of an ablation electrode for radiofrequency catheter ablation. The tip 61 may be electrically connected with an electrode wire 33 and may be heated with externally applied power to remove cardiac muscular tissue. In another embodiment, the tip 61 may be implemented as an electrical sensor element capable of measuring a bio-signal such as ECG. The tip 61 is coupled to a front end of the catheter body 63. One or more driving wires 615 are connected to the tip 61, such that the catheter 6 may be steered as a direction of the front end is controlled via inflow and outflow of the driving wires 615. Water supply holes 613 may be defined in an outer surface of the tip 61. Coolant delivered to an irrigation tube 31 may be discharged through the water supply holes 613.

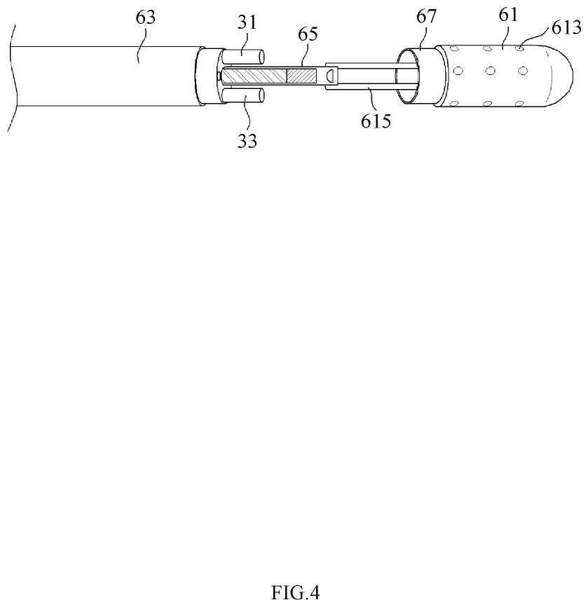

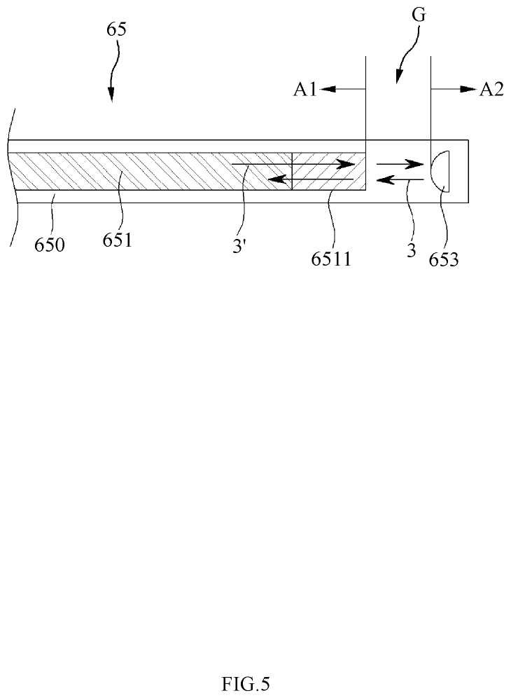

In the catheter body 63, a path in which one or more channels are formed as a first region (A1, FIG. 4), while a front end having the tip 61 to which the external force is applied is defined as a second region (A2, FIG. 5). A gap G may be defined between the first region (A1, FIG. 5) and the second region (A2, FIG. 5).

The catheter body 63 enters the heart and guides, to a target site, a treatment instrument such as the electrode which must be inserted to remove the cardiac muscular tissue. In treatment of tachyarrhythmias, such as paroxysmal atrophy, atrial tachycardia, and paroxysmal ventricular tachycardia, the heated electrode comes into contact with the tissue to remove the cardiac muscular tissue. The electrode performs ablation for about 60 seconds at about 50 to 60° C. As such, a catheter in which the electrode reaches the arrhythmia site and removes the cardiac muscular tissue to treat the arrhythmia may be classified as an ablation catheter. The electrode may be provided for measuring the bio-signal in addition to the removal of the cardiac muscular tissue. Depending on a purpose of the treatment and a surgical procedure method, a treatment instrument such as a stent may be guided. The catheter body 63 is made of a highly biocompatible and flexible material to allow the electrode or other treatment instrument of a front end used in the ablation catheter or a mapping catheter to be guided to a target site.

FIG. 4 shows an exploded view of the front end of the catheter 6 according to an embodiment of the present disclosure. FIG. 5 shows an internal structure of the optical fiber 65 of the catheter 6 according to an embodiment of the present disclosure.

One or more channels may be formed in the catheter body 63. Referring to FIG. 4, it is shown that in an embodiment of the channels formed in the catheter body 63, a channel for receiving the optical fiber 65 for the pressure measurement, a channel for receiving the irrigation tube 31 for cooling the heated electrode, a channel for receiving the electrode wire 33 for supplying power to the electrode, and a channel for receiving the driving wire 615 for steering of the catheter 6 are formed.

Referring to FIG. 5, as used herein, the front end provided with the tip 61 of the catheter body 63 is divided into the first region A1 and the second region A2. Those regions are defined to clearly describe a structural feature and a function of the configuration. The first region A1 extends from the front end of the catheter 6 to a path of the catheter body 63 where the optical core 651 is located. The second region A2 extends a reflective mirror 653 to the tip 61. A space between the first region A1 and the second region A2 is referred to as the gap G.

The configuration of the catheter 6 according to the present embodiment to be described later may measure the external force in consideration of the direction using the gap G of a single step. The second region A2 after the gap G is bent along the direction of the external force. Thus, the optical core 651 receives the reflective light so as to discriminate a bending direction and a degree of bending between the first region A1 and the second region A2.

The optical fiber 65 includes the optical core 651 that is received into the channel of the catheter body 63 and is located in the first region A1. As the optical core 651 emits light to the second region A2 through the gap G, light reflected from the reflective mirror 653 may be received by the optical core 651. The optical fiber 65 may be implemented such that a sheath 650 shields the optical core 651. A clad layer may be formed inside the sheath 650 to allow light to be delivered through the optical core 651 in a total reflection manner.

In this embodiment, the optical core 651 emits incident light to the reflective mirror 653. Reflective light reflected from the reflective mirror 653 is received by the optical core 651. As described above, the amount information of the reflective light that the optical core 651 receives may vary according to a degree to which the front end of the catheter body 63 is bent or pressed. This is due to a structural feature of the reflective mirror 653 as described below.

In this embodiment, the optical fiber 65 has an optical filter 6511 coated on an output end thereof. The optical filter 6511 reflects a portion of the light emitted from the optical core 651 to the gap G therefrom, and transmits the remaining portion thereof therethrough to the second region A2 to allow only a portion of the light irradiated from the optical core 651 to be emitted to the second region A2. In this embodiment, the optical filter 6511 may be made of a crystal material. Depending on an unique nature of the material, the optical filter may transmit light of a specific wavelength and reflect light of a specific wavelength.

In this embodiment, the amount information of light leaked from the gap G of the front end may be understood as a main variable for quantifying the pressure information of the front end. However, the optical core 651 has a characteristic that light transmitted to the inside of the optical core 651 is lost due to temperature change or bending. That is, a proximal portion of the catheter body 63 inevitably bends as it enters the heart. In this connection, the reflective light is lost due to the bending occurring in the first region A1. Thus, the amount information of the light lost in the first region A1 and the lost amount information of the light reflected in the reflective mirror 653 of the second region A2 may not be distinguished between each other. Therefore, the light-amount information lost as the front end as the distal portion of the catheter body 63 is bent and the light-amount information lost within the optical core 651 as the proximal portion of the catheter body 63 is bent should be distinguished from each other to set a reference value. Note that, according to this need, the optical filter 6511 which transmits only light of a specific wavelength therethrough should be coated on the front end of the optical core 651.

Thus, the optical fiber 65 receives the light reflected from the optical filter 6511 and defines the light as first light 3′ and receives the light transmitted through the optical filter 6511 and reflected by the reflective mirror 653 and defines the light as second light 3. The first light 3′ may be defined as light of a wavelength band as reflected from the optical filter 6511. The second light 3 may be defined as light of a wavelength band passing through the optical filter 6511.

As a result, the first light 3′ reflects the change of the amount of reflective light according to the bending of the optical core 651 or the temperature change. The second light 3 reflects the change in the amount of reflective light received by the optical core 651 according to the displacement of the reflective mirror 653. The light-amount analyzer 8 senses the direction and magnitude of the external force applied to the tip 61 using the light-amount information of the second light 3.

The reflective mirror 653 is provided inside the front end and located in the second region A2 and is configured so that a spherical surface thereof is not flat toward the first region A1. The reflective mirror 653 is spaced apart from the output end of the optical core 651 by a spacing of the gap G and is positioned in the second region A2 whose bending occurs relative to the first region A1 about the gap G.

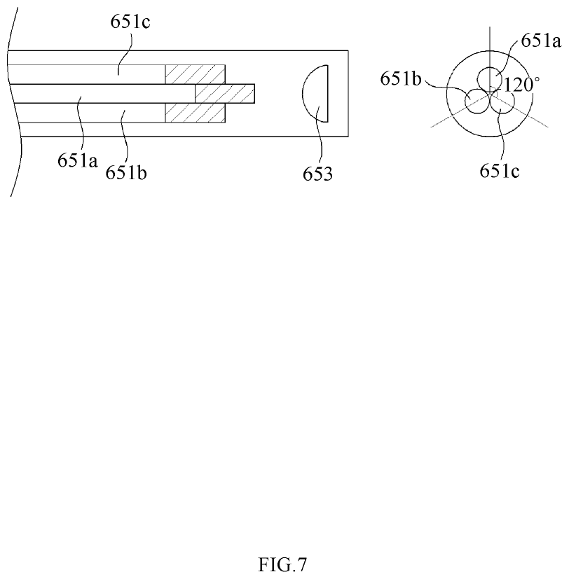

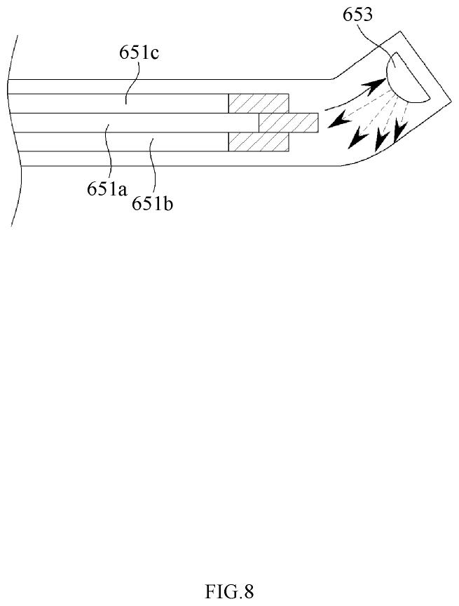

In the present embodiment, the reflective mirror 653 has a convex surface toward the first region A1. When the external force is applied outwardly from the linear axis of the catheter body 63, the light output from the optical core 651 is incident on the reflective mirror 653 at an oblique angle. That is, the reflective mirror 653 has a convex spherical surface toward the output end of the optical core 651. When the reflective mirror 653 is displaced by the external force, a portion of the output light may be re-input into the optical core 651. Further, in a structure of multiple optical cores 651, which will be described later in FIG. 7 and FIG. 8, the convex sphere of the reflective mirror 653 allows the amounts of light which the optical cores 651 may receive respectively to be distinguished from each other.

The elastic member 67 is provided to surround the gap G and is disposed inside the catheter body 63 and is made of a material having a different elastic force from that of the catheter body 63, so that the external force applied to the tip 61 may be concentrated on the front end.

The catheter 6 is most preferably implemented as a single piece of a single material so that there is no step. However, in order to accurately measure the magnitude and direction of the external force applied to the tip 61, it is required that a heterogeneous material with a different elastic force from that of the catheter body 63 is provided at the front end. In this embodiment, a spacing of the gap G formed in the optical fiber 65 and the displacement of the reflective mirror 653 act as the main factors for measuring the magnitude and direction of the external force applied to the tip 61. Therefore, it is necessary that the external force exerted on the tip 61 is accurately associated with the displacement of the gap G. Eventually, when the catheter body 63 made of the same elastic material extends to the gap G, an external force in the axial direction is applied to the tip 61 of the front end but most of the external force is transmitted to the catheter body 63. Thus, a precise displacement of the gap G may not occur. Further, the external force is applied outwardly to the tip 61 of the front end but the bending is not concentrated on a region adjacent to the gap G, thus making it difficult to accurately measure the change in the light-amount. For this reason, it is desirable to provide the elastic member 67 made of a heterogeneous material and assembled to the front end of the catheter body 63 together with the tip 61 and surrounding the gap G located inside the catheter body 63. The elastic member 67 may be made of a more flexible material than the material of the catheter body 63. In an example, the elastic member 67 is embodied as an element such as a spring.

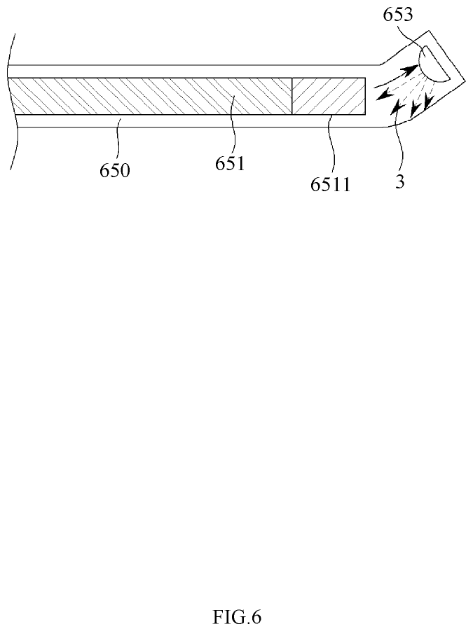

FIG. 6 shows an internal structure of the optical fiber when an external force is applied upwardly to the front end of the catheter 6 according to an embodiment of the present disclosure. FIG. 6 shows a case where when an external force is applied outwardly (upwardly) from the linear axis of the catheter body 63, the light output from the optical core 651 is incident on the reflective mirror 653 at an oblique angle.

Referring to FIG. 6, the reflective mirror 653 is located in the second region A2. Therefore, when the external force is applied to the tip 61, the second region A2 is bent and thus the reflective mirror 653 is displaced upwards. When the reflective mirror 653 is pressed and displaced upwards, the light output to the reflective mirror 653 is incident at an oblique angle onto an interface of the mirror, so that only a portion of the output light is received by the optical core 651 back. As a result, the optical fiber 65 receives the second light 3 at a significantly reduced light-amount. Unlike FIG. 6, when the external force is applied in the linear axis direction of the catheter body 53, the reflective mirror 653 moves toward the optical core 651 without the tilting and thus the size of the gap G is reduced. In this case, the amount of reflection of the second light 3 reflected from the reflective mirror 653 is increased so that the optical fiber 65 receives the second light 3 at an increased light-amount. However, as shown in FIG. 6, the sensing assembly of a single optical core 651 may only distinguish between the linear axial directional pressing and the pressing outwardly from the linear axis.

Therefore, the catheter 6 according to the present embodiment has three or more optical cores 651, so that the light-amounts may be obtained based on three or more outwards directions relative to the linear axis. FIG. 7 shows an internal structure of an optical fiber with three or more optical cores 651 according to another embodiment of the present disclosure. FIG. 8 shows an internal structure of the optical fiber 65 when an external force is applied upwards to the front end of the catheter 6 according to the embodiment of FIG. 7.

Referring to FIG. 7 and FIG. 8, in an embodiment, multiple optical cores 651a, 651b, and 651c are provided in a single optical fiber 65. Alternatively, three optical fibers 65, each having a single optical core 651 may be provided in in the catheter body 63.

In the optical fiber 65 according to the present embodiment, the three optical cores 651 are arranged at an angular spacing of 120°. Thus, different amounts of the reflected light from the reflective mirror 653 as displaced laterally as the lateral directional external force is applied to the tip 61 may be received by the three optical cores 651a, 651b, and 651c respectively.

As shown in FIG. 8, when the reflective mirror 653 is pressed upwards and displaced upwards, the first optical core 651c receives the second light 3 at the smallest light-amount, the second optical core 651a receives the second light 3 at a certain light-amount, and the third optical core 651b receives the second light 3 at the largest light-amount. Thus, the multiple optical cores 651a, 651b, and 651c which are arranged at the 120° spacing may distinguish between the light-amounts of the second light 3 as received from the reflective mirror 653 based on the tilted displacement. Thus, three-dimensional directionality of the external force may be considered.

Further, the three optical cores 651a, 651b, and 651c should distinguish between the light-amounts of the second light as variables. For this reason, light of different wavelength bands may be incident on the three optical cores 651a, 651b, and 651c respectively. In an example, light of R, G, and B wavelengths may be incident on the three optical cores 651a, 651b, and 651c, respectively. Comparing the light-amount of the red wavelength, the light-amount of the green wavelength, and the light-amount of the blue wavelength with each other may allow the three-dimensional directionality in which the external force is applied to the tip to be determined. Alternatively, in another embodiment, light may be incident on the three optical cores 651a, 651b, and 651c at different timings.

In another embodiment, the optical fiber 65 may include four optical cores. In this embodiment, the four optical cores are arranged at 90° spacing, so that the four optical cores respectively different amounts of light received from the reflective mirror 653 as laterally displaced as the lateral external force is applied to the tip 61. As such, the optical fiber 65 may include a plurality of optical cores. That is, the optical fiber may include at least three optical cores.

The light-amount analyzer 8 may include the processor 81 and the display 83.

The light-amount analyzer 8 receives the amount of the reflected light received by the optical fiber 65 and calculates the direction and magnitude of the external force applied to the tip 61 based on change in the amount of the light. The light-amount analyzer 8 receives the amount of the first light 3 as the light reflected from the optical filter 6511, and receives the amount of the second light 3 as the light reflected from the reflective mirror 653 through the optical filter 6511, and uses the light-amount information of the second light 3 to calculate the direction and magnitude of the external force applied to the tip 61 of the catheter 6. The light-amount analyzer 8 injects light of varying wavelength bands or at a varying timings to discriminate light-amount information from the multiple optical cores 651a, 651b, and 651c. The processor 81 may calculate the change in the amount of the received second light 3. The display 83 may visually display the change in the light-amount.

As described above, according to the present embodiment, the catheter is provided to measure, with high sensitivity, the magnitude and direction of the external force applied to the front end of the catheter 6 using the change information of the amount of light received by the optical fiber 65 in the catheter body 63. In particular, the catheter 6 according to the present embodiment is configured to measure the external force of the front end using only the single gap G structure defined between the first region A1 and the second region A2 in the front end of the catheter body 63. Therefore, the sensing assembly may be implemented in a small region of the front end the catheter 6. Further, the light-amount analyzer 8 measures the pressure value based on analysis of the change of the light-amount. The light-amount information may be easily acquired and analyzed. Thus, it is not difficult to design a system for pressure sensing. Further, a manufacturing cost thereof may be reduced. The light-amount analyzer 8 may determine the direction in which the pressure is applied to the tip based on the light-amount information from the at least three optical cores 651a, 651b, and 651c arranged at the 120° spacing. When the external force is applied to the catheter tip 61, the reflective mirror 653 may be displaced in a tilted manner in the direction of the external force, so that the light-amounts received by the three optical cores 651a, 651b, and 651c are distinguishable from each other. In particular, the structure of the reflective mirror 653 has the spherical surface having a curvature, and thus the mirror reflects the output light from the optical cores 651a, 651b, 651c at an oblique angle when the mirror is displaced in a laterally tilted manner. Accordingly, the three optical cores 651a, 651b, and 651c receive remarkably reduced light-amounts when a lateral pressure is applied than when a pressure in a linear axial direction of the catheter is applied, thereby discriminating the direction of the external force.

Although the present disclosure has been described in detail based on the representative embodiments above, those skilled in the art to which the present disclosure belongs will understand that various modifications may be made without departing from the scope of the present disclosure. Therefore, the scope of the present disclosure should not be limited to the embodiments as described but should be determined based on the claims to be described later and all changes or modifications derived from the claims and the equivalent concepts.

1. A catheter comprising:
a catheter body having first and second regions, wherein one or more channels are formed in the first region, while the second region comprises a front end having a tip subjected to an external force, wherein a gap is defined between the first region and the second region;
an optical fiber comprising an optical core received in the channel and located in the first region, wherein the optical core emits light through the gap into the second region and then receives light reflected from a reflective mirror; and
the reflective mirror located inside the front end and located in the second region, wherein the reflective mirror is positioned such that a spherical face of the mirror is not flat toward the first region,
wherein as the external force is applied to the tip, a spacing in the gap between an output end of the optical core and the reflective mirror varies, and
wherein the catheter senses a direction and a magnitude of the external force applied to the tip based on change in an amount of light reflected from the reflective mirror due to the variation.

2. The catheter of claim 1, wherein the catheter further comprises an elastic member provided to surround the gap and disposed inside the catheter body and made of a material having an elastic force different from an elastic force of the catheter body, such that the elastic member concentrates the external force applied to the tip on the front end.

3. The catheter of claim 1, wherein the optical fiber comprises three optical cores arranged at an angular spacing of 120°, wherein the three optical cores respectively receive different amounts of light reflected from the reflective mirror displaced laterally as a lateral external force is applied to the tip.

4. The catheter of claim 1, wherein the optical fiber further comprises an optical filter coated on the output end thereof, wherein the optical filter reflects a portion of light emitted from the optical core into the gap and transmits a remaining portion of the light emitted from the optical core therethrough to the second region.

5. The catheter of claim 4, wherein the optical fiber receives the light reflected from the optical filter as first light, and receives the light reflected from the reflective mirror through the optical filter as second light, and
wherein the catheter senses the direction and the magnitude of the external force applied to the tip based on amount information of the second light.

6. The catheter of claim 1, wherein the reflective mirror has a spherical face convex toward the first region, and
wherein when the external force is applied outwardly from an linear axis of the catheter body, light output from the optical core is incident on the reflective mirror at an oblique angle.

7. A catheter system comprising:
a catheter comprising:
a catheter body having first and second regions, wherein one or more channels are formed in the first region, while the second region comprises a front end having a tip subjected to an external force, wherein a gap is defined between the first region and the second region;
an optical fiber comprising an optical core received in the channel and located in the first region, wherein the optical core emits light through the gap into the second region and then receives light reflected from a reflective mirror; and
the reflective mirror located inside the front end and located in the second region; and
a light-amount analyzer to receive an amount of the reflected light received by the optical fiber and to calculate a direction and a magnitude of the external force applied to the tip based on change in the received amount of the light.

8. The catheter system of claim 7, wherein the optical fiber further comprises an optical filter coated on the output end thereof, wherein the optical filter reflects a portion of light emitted from the optical core into the gap and transmits a remaining portion of the light emitted from the optical core therethrough to the second region, and
wherein the light-amount analyzer receives an amount of first light as the light reflected from the optical filter, and an amount of second light as the light reflected from the reflective mirror through the optical filter and calculates the direction and magnitude of the external force applied to the tip of the catheter based on the amount of the second light.
